(19) United States
(12) Patent Application Publication
(10) Pub. No.: US 2008/0057096 A1
(43) Pub. Date: Mar. 6, 2008

(54) Biocompatible stent

(75) Inventor: Robert L. Ibsen, Santa Maria, CA (US)

Correspondence Address: CROWELL & MORING LLP;INTELLECTUAL PROPERTY GROUP, P.O. BOX 14300, WASHINGTON, DC, 20044-4300, US

(73) Assignee: DEN-MAT CORPORATION

(21) Appl. No.: 11/511,509

(22) Filed: Aug. 29, 2006

(51) Int. Cl. A61F 2/02; A61K 48/00; A61K 38/18; A61K 31/727; A61K 31/60

(52) U.S. Cl. 424/423; 514/56; 514/171; 514/165; 514/12; 514/44; 424/673

(57) ABSTRACT

A biocompatible stent with a fluoride-containing hydrophilic water insoluble crosslinked resin layer. A therapeutic substance-containing layer may also be included. A method of making a biocompatible stent.

### BACKGROUND

In percutaneous transluminal coronary angioplasty (PTCA), a procedure for treating heart disease, a catheter assembly having a balloon portion is introduced percutaneously into the cardiovascular system of a patient via the brachial or femoral artery. The catheter assembly is advanced through the coronary vasculature until the balloon portion is positioned across the occlusive lesion. The balloon is then inflated to a predetermined size to radially compress against the atherosclerotic plaque of the lesion to remodel the lumen wall. The balloon is then deflated to a smaller profile to allow the catheter to be withdrawn from the patient's vasculature.

One problem associated with the above procedure includes the development of thrombosis and restenosis of the artery over several months after the procedure, which may require another angioplasty procedure or a surgical by-pass operation. A stent is often implanted in the lumen to maintain the vascular patency.

Even with the use of a stent, restenosis can still occur and the stent may itself exacerbate the thrombosis. To prevent restenosis, stents are commonly coated with a biocompatible material such as fibrin that reduces the adhesion of proteins in the blood to the stent. To prevent and treat the restenosis and thrombosis, various pharmacological agents may be included in the stent. The agent is then eluted from the stent over a period of time to provide local administration to the artery at the site of the stent. In order to combine these effects, stents are commonly provided with a drug eluting layer covered by a biocompatible layer.

The present invention provides a novel biocompatible layer that also leaches fluoride to provide further therapy to the patient's body at the site of the stent. The biocompatible layer can be used by itself or in conjunction with a drug eluting layer.

### SUMMARY

One embodiment of the invention includes a biocompatible drug eluting stent. The stent includes a first layer and a second layer. The first layer includes a therapeutic substance. The second layer includes a fluoride-containing hydrophilic water insoluble crosslinked resin.

Another embodiment of the invention includes a method for forming a biocompatible drug eluting stent. A first layer is provided to a stent body. The first layer includes a therapeutic substance. A second layer is then provided over the first layer. The second layer includes a fluoride-containing hydrophilic water insoluble crosslinked resin.

Another embodiment of the invention includes a method for forming a biocompatible stent. A fluoride-containing hydrophilic water insoluble crosslinked resin layer is provided to a stent body.

Another embodiment of the invention includes a biocompatible drug eluting stent. The stent includes a stent body. The stent body is covered with an inner layer, an intermediate layer over the inner layer and an outer layer over the intermediate layer. A fluoride-containing hydrophilic water insoluble crosslinked resin is in both the inner and outer layers. A therapeutic substance is in the intermediate layer.

Another embodiment of the invention includes a biocompatible drug eluting stent. The stent includes a stent body. The stent body is covered with a fluoride-containing hydrophilic water insoluble crosslinked resin. A therapeutic substance is incorporated within the resin.

### BRIEF DESCRIPTION OF THE DRAWINGS

FIG. 1A is a cross-sectional representation of a fragmentary portion of a biocompatible drug eluting stent of one embodiment of the present invention.

FIG. 1B is a cross-sectional representation of a fragmentary portion of a biocompatible drug eluting stent including a primer coating of another embodiment of the present invention.

FIG. 2A is a cross-sectional representation of a fragmentary portion of a biocompatible stent of another embodiment of the present invention.

FIG. 2B is a cross-sectional representation of a fragmentary portion of a biocompatible stent including a primer coating of another embodiment of the present invention.

FIG. 3A is a cross-sectional representation of a fragmentary portion of a biocompatible drug eluting stent including two biocompatible layers of another embodiment of the present invention.

FIG. 3B is a cross-sectional representation of a fragmentary portion of a biocompatible drug eluting stent including two biocompatible layers and a primer coating of another embodiment of the present invention.

FIG. 4A is a cross-sectional representation of a fragmentary portion of a biocompatible drug eluting stent including a therapeutic substance incorporated into a biocompatible layer of another embodiment of the present invention.

FIG. 4B is a cross-sectional representation of a fragmentary portion of a biocompatible drug eluting stent including a therapeutic substance incorporated into a biocompatible layer and a primer coating of another embodiment of the present invention.

FIG. 5 is a graph showing the optional fluoride leachable property of the fluoride-containing layer in one embodiment of the invention.

FIG. 6 is an area plot of concentration of a siliceous fluoride source used in one embodiment of the invention.

### DESCRIPTION

It is to be understood that the forgoing figures are for illustrative purposes only and are not meant to limit the scope or breadth of the invention in any manner. The figures are not drawn to scale and the thickness of different layers is purely for illustration.

One embodiment of the instant invention includes a biocompatible drug eluting stent. The stent includes a first layer having a therapeutic substance. The stent also includes a second layer comprising a fluoride-containing hydrophilic water insoluble crosslinked resin.

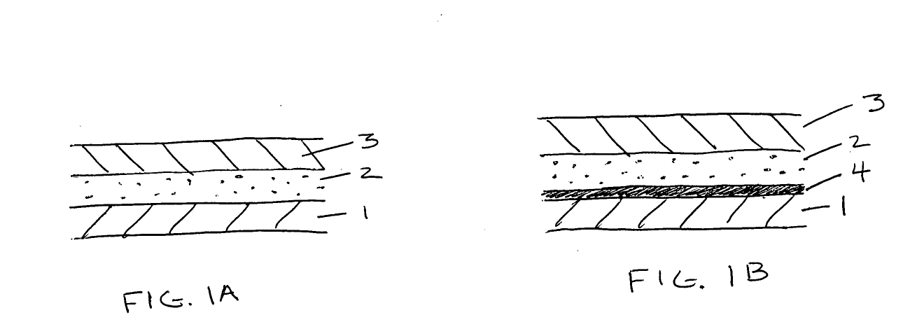

FIG. 1A shows a cross sectional view of a fragmentary portion of a biocompatible drug eluting stent according to this embodiment. A therapeutic substance containing layer 2 lies over stent body 1. A fluoride-containing hydrophilic water insoluble crosslinked resin layer 3 lies over the therapeutic substance containing layer 2. FIG. 1B shows an optional primer coating between stent body 1 and therapeutic substance containing layer 2.

Another embodiment of the instant invention includes a biocompatible stent. The stent includes a covering of a fluoride-containing hydrophilic water insoluble crosslinked resin.

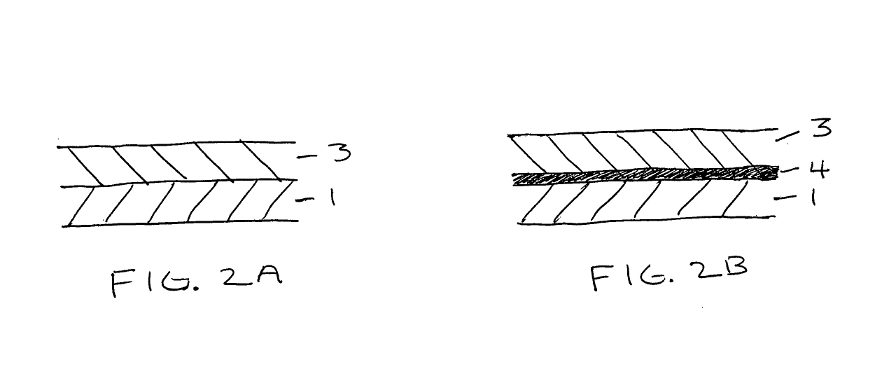

FIG. 2A shows a cross sectional view of a fragmentary portion of a biocompatible stent according to this embodiment. A fluoride-containing hydrophilic water insoluble crosslinked resin layer 3 lies over stent body 1. FIG. 2B shows an optional primer coating between stent body 1 and fluoride-containing hydrophilic water insoluble crosslinked resin layer 3.

Another embodiment of the instant invention includes a biocompatible drug eluting stent. The stent includes a therapeutic substance containing layer sandwiched between two fluoride-containing hydrophilic water insoluble crosslinked resin layers.

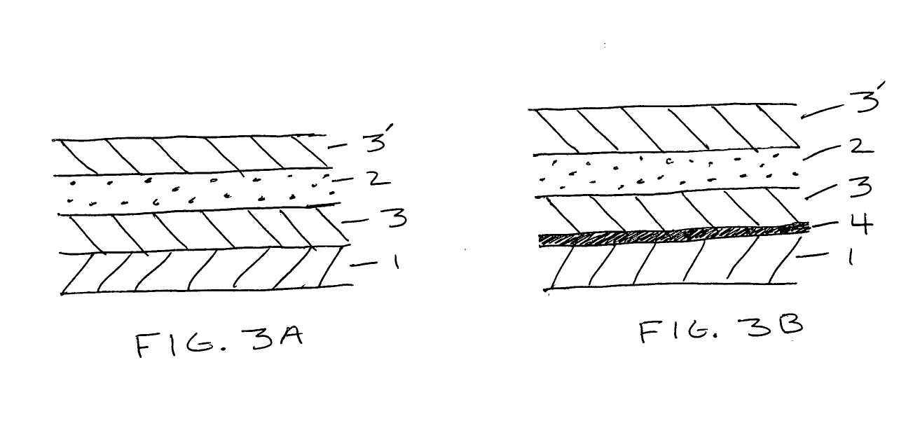

FIG. 3A shows a cross sectional view of a fragmentary portion of a biocompatible drug eluting stent according to this embodiment. A fluoride-containing hydrophilic water insoluble crosslinked resin layer 3 lies over stent body 1. A therapeutic substance containing layer 2 lies over fluoride-containing hydrophilic water insoluble crosslinked resin layer 3. Another fluoride-containing hydrophilic water insoluble crosslinked resin layer 3′ lies over the therapeutic substance containing layer 2. Fluoride-containing hydrophilic water insoluble crosslinked resin layer 3′ may be made of the same or different material as fluoride-containing hydrophilic water insoluble crosslinked resin layer 3. FIG. 3B shows an optional primer coating between stent body 1 and fluoride-containing hydrophilic water insoluble crosslinked resin layer 3.

Another embodiment of the instant invention includes a biocompatible drug eluting stent. The stent includes a therapeutic substance dispersed throughout a fluoride-containing hydrophilic water insoluble crosslinked resin coating.

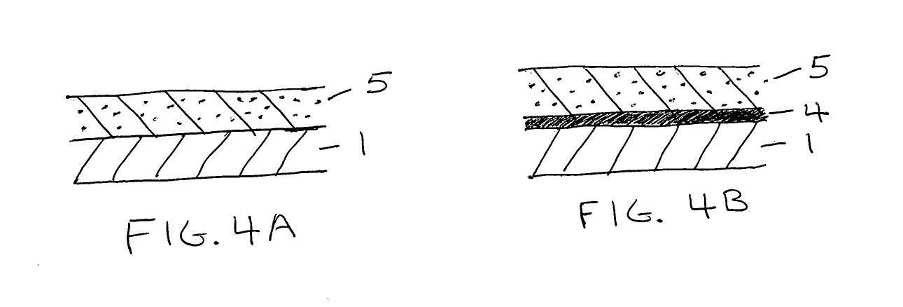

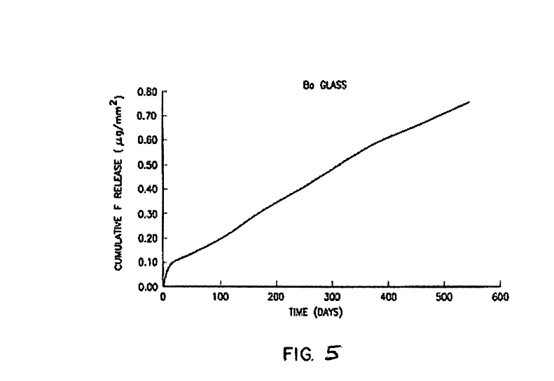

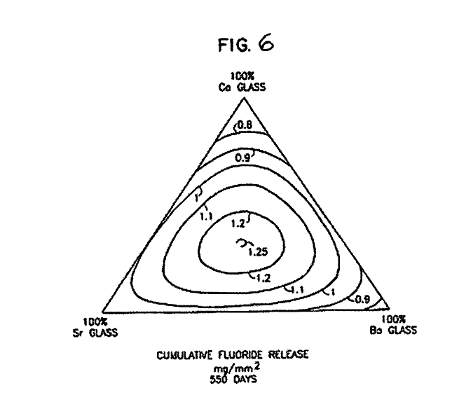

FIG. 4A shows a cross sectional view of a fragmentary portion of a biocompatible drug eluting stent according to this embodiment. A layer 5 of a fluoride-containing hydrophilic water insoluble crosslinked resin in which a therapeutic substance is incorporated into lies over stent body 1. FIG. 4B shows an optional primer coating between stent body 1 and layer 5.

The therapeutic substance may include any substance that is known to be effective against restenosis or thrombosis or any other substance that is known to be included in drug eluting stents. Exemplary substances include anticoagulant drugs, antiplatelet drugs, antimetabolite drugs, anti-inflammatory drugs and antimitotic drugs such as glucocorticoids (e.g. dexamethasone, betamethasone), heparin, hirudin, tocopherol, angiopeptin, aspirin, ACE inhibitors, growth factors, and oligonucleotides. Of course any drug whose elution is known to be advantageous at the site of a stent may be included.

The fluoride-containing hydrophilic water insoluble crosslinked resin coating preferably comprises the compound known as Geristore™ (or some variation thereof), sold by Den-Mat Corporation and disclosed in U.S. Pat. Nos. 4,738,722, 5,334,625, 5,151,543, and 5,876,743, each of which is hereby incorporated by reference in its entirety. A description of the coating and method for forming it follows.

The coating is typically a crosslinked heat and/or light set resin that contains hygroscopic groups that attract water to the coating. When the crosslinking is not too extensive, the coating can absorb enough water that it can swell. The amount of water that the coating can absorb can be as high as 37 weight percent. However, the degree of crosslinking of the coating is typically high enough that water absorption (determined according to ADA Specificaton No. 27) will not exceed about 10 weight percent, preferably not exceeding about 7 weight percent. The backbone of the polymer providing the hygroscopic groups of the resin phase of the coating is typically aliphatic and may contain groups therein that enhance the hydrophilicity of the resin phase. Though the coating's resin can be made by a condensation reaction, such as by low temperature resin formation by the reaction of a blocked polyisocyanate with a polyol, the resin is typically the in situ reaction product of one or more of a polymerizable ethylenically unsaturated organic monomer containing groups that are attractive to water. Thus, the coating may contain the following components:

(a) an ethylenically unsaturated-functional monomer that contains a hygroscopic group. Typical of such groups are hydroxyl, amide, amine, aliphatic ether, amine, hydroxyalkyl amine, hydroxyalkyl amide, pyrrolidone, ureyl, and the like. Illustrative of such monomers are the following:

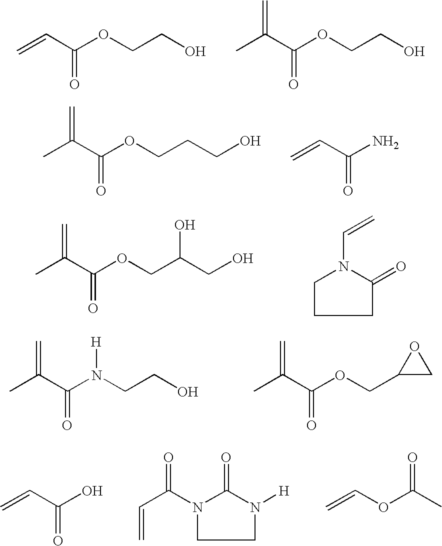

A particularly desirable ethylenically unsaturated-functional monomer is an acrylic-type monomer having the following structure:

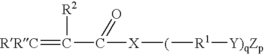

wherein R′ and R″, individually, are hydrogen, alkyl of 1 to about 4 carbon atoms, monocyclic aryl, such as phenyl, alkyl phenyl where the alkyl is 1 to about 3 carbon atoms, cyclohexyl, and the like; R² is hydrogen, alkyl of 1 to about 3 carbon atoms, and the like; X is O, S and N—R³, where R³ is hydrogen, alkyl of 1 to about 4 carbon atoms, —R¹-Y, and the like; R¹ is a divalent radical connecting Y to X, and may be one of the following:

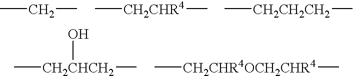

wherein each R⁴ is hydrogen or alkyl of 1 to about 3 carbon atoms; and Y is OH, NR⁵, SH, OR⁶, where R⁵ is hydrogen, methylol, methylol methyl ether, R⁶ is alkyl of 1 to about 3 carbon atoms provided that R¹ is —CH²—, and the like; q is 0 or 1 and p is 0 or 1, and p is 0 when q is 1 and 1 when q is 0; Z is hydrogen.

A particularly desirable thermosetting coating is based on 2-hydroxyethyl methylmethacrylate (“HEMA”), 2-hydroxyethyl acrylate, 2,3-dihydroxypropyl methacrylate, acrylamide, methacrylamide, hydroxyalkyl acrylamide, hydroxyalkyl methacrylamide, and the like materials.

(b) A linear polycarboxylic acid or acid salt that contains a plurality of pendant carboxyl or carboxylic acid salt groups such as one having the formula:

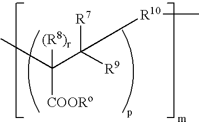

R⁰ is hydrogen or alkali metal, such as Li, Na, K, Ru and Cs to form a salt, and preferably hydrogen, sodium or potassium, R⁷ and R⁸ are hydrogen or alkyl containing from 1to about 3 carbon atoms, R⁹ is hydrogen, alkyl of 1 to about 3 carbon atoms, or COOR∘, provided that R⁹ is not alkyl when R⁷ is alkyl, R¹⁰ is a valence bond when the formula is for a homopolymer or a divalent organic moiety of a polymerized ethylenically unsaturated monomer, p is a number representing at least 40 mole percent of the units of the polymer, and m is a number providing for a molecular weight of from about 2,000 to about 500,000. Particularly preferred polycarboxylic acids are polyacrylic acid, polymaleic acid, polyitaconic acid, or a copolymer of acrylic acid, maleic acid, fumaric acid or itaconic acid with other ethylenically unsaturated monomers such as methyl acrylate, ethylacrylate, methylmethacrylate, vinyl acetate, vinylmethylether, styrene, α-methylstyrene, vinylcyclohexane, dimethylfumarate, ethylene, and the like. Preferably, these polymers have molecular weights M_{w} of about 3000-250,000. In one embodiment, the polycarboxylic acid or the salt form may contain about 1-5 weight % of d-tartaric acids.

(c) A desirable coupling agent is an acrylic-type monomer that possesses acrylic-type unsaturation and contains a surface bonding group possessing one or more of the following groups:

| | |
|---|---|
| i) | an alkylene polyether; |
| ii) | hydroxyl |
| iii) | carboxyl |
| iv) | carboxylic acid salt |
| v) | quaternary ammonium |
| vi) | tertiary amine |
| vii) | phosphoryl |
| viii) | phosphinyl |
| ix) | stannoyl |
| x) | amide |
| xi) | alkylene amine |

A preferred coupling agent is a simple aromatic substituted amino acid or its alkali metal salt such as the free acid or alkali metal salt of (i) N-phenylglycine, (ii) the adduct of N-(p-tolyl)glycine and glycidyl methacrylate, which are illustrated by the structures:

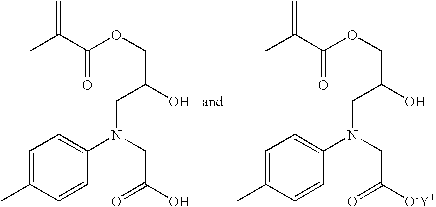

where Y is one of the alkali metals, i.e., lithium, sodium, potassium, rubidium and cesium, preferably sodium or potassium, and (iii) the adduct of N-phenylglycine and glycidyl methacrylate, the alkali metal salt thereof, or the mixture of the foregoing two compounds, which compounds are illustrated by the structures, and (iii) the adduct of N-phenylglycine and glycidyl methacrylate, which are illustrated by the structures:

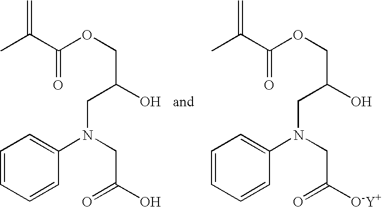

where Y is described above; or the mixture of the foregoing two compounds, alone or in combination with a compound containing at least one group or moiety capable of free radical polymerization and at least one aromatic ring or moiety containing one or more electron-withdrawing substituent that does not interfere with free radical polymerization.

The purpose of the coupling agent is to interreact with the polymerization of the aforementioned ethylenically unsaturated-functional monomer that contains a hygroscopic group and enhance wetting by the resulting resin of proteinaceous surfaces by the surfaces interaction with the carboxylic acid or carboxylic acid salt group in the bonding agent.

(d) A number of acrylic coating resins rely on polyacrylyl substituted monomers to crosslink and chain extend the polymer that comes into existence on polymerization in the presence of an polymerization initiator. For example, the pure forms of HEMA typically contain small amounts of ethylene glycol dimethacrylate which will crosslink a polymer based on HEMA. The degree of crosslink may be so minuscule as to have little effect on the ultimate properties of the polymer. Crosslinking agents are frequently added to HEMA based resins to impart a particular quality of crosslinking and toughness to the cured resin. For example, diethylene glycol dimethacrylate can otherwise lower the crosslink density of the resin which may impart toughness to the resulting cured polymer. Those types of crosslinkers would be considered a soft crosslinker, as defined above. However, in the practice of this invention, it is desired to use dual crosslinkers, one that is hard and one that is soft.

In this respect, one may include the above crosslinker, in its normal impurity concentrations, as part of the soft crosslinker, but in the preferred embodiment, it is desirable to employ hard and soft crosslinkers that contain at least two acrylyl groups bonded to aromatic containing moiety(ies). A desirable hard crosslinker is characterized by the following formulae:

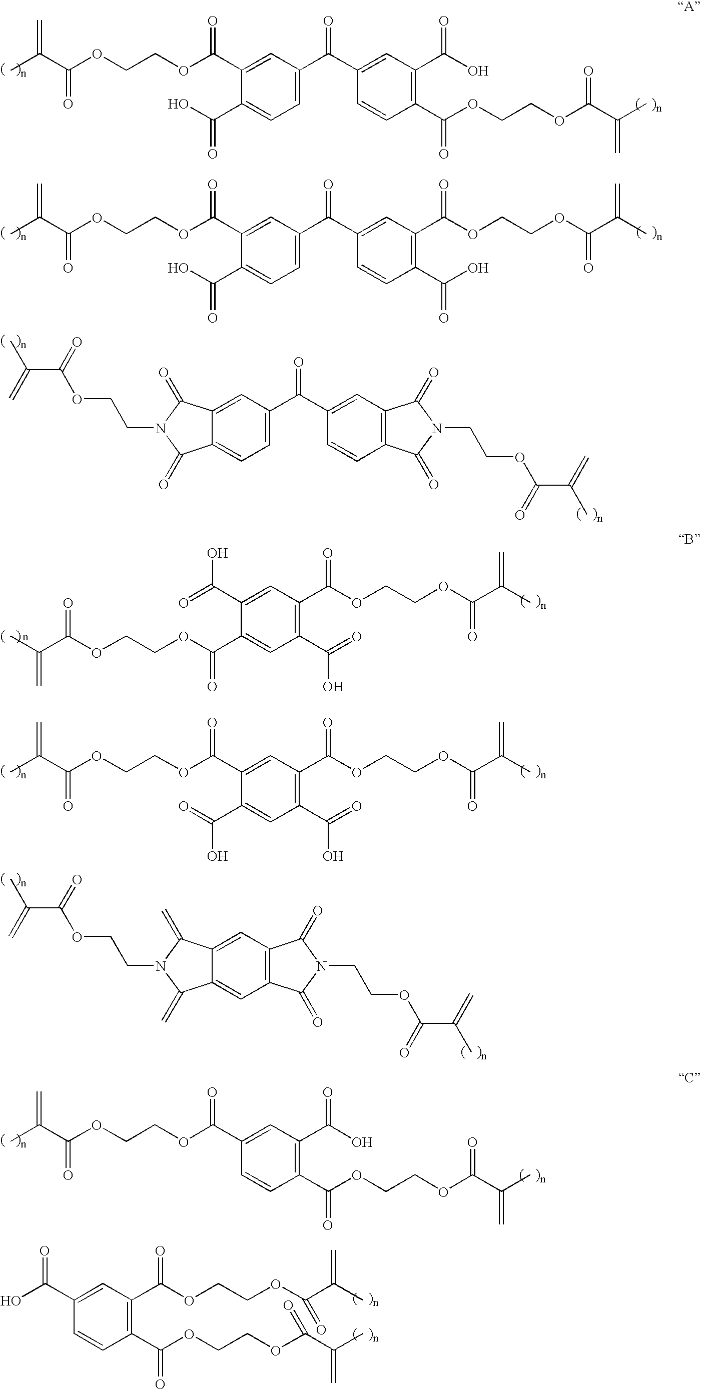

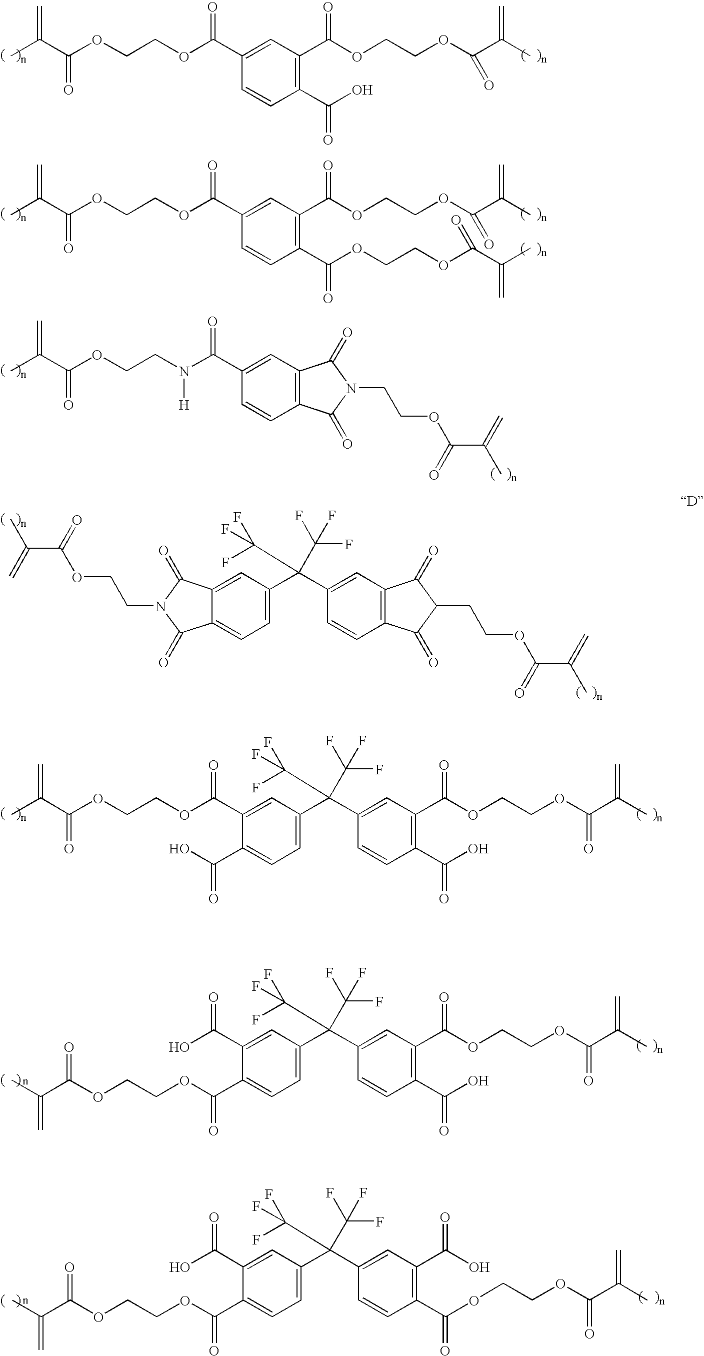

wherein n is 0 or 1. The preferred hard crosslinking agent is one of (i) the esters or imides of pyromellitic acid dianhydride and 2-hydroxyethyl methacrylate or 2-aminoethyl methacrylate, or the corresponding acrylates, as illustrated in group B above, (ii) the ester or imides of 3,3′, 4,4′-benzophenonetetracarboxylic dianhydride and 2-hydroxyethylmethacrylate or 2-aminoethyl methacrylate, or the corresponding acrylates, as illustrated in group A above, (iii) the esters and imide/amides of 4-trimellitic acid anhydride and 2-hydroxyethylmethacrylate or 2-aminoethyl methacrylate, or the corresponding acrylates, as illustrated in group C above, (iv) the ester or imides of 2,2-bis(3,4,-dianhydridophenyl)-1,1,1,3,3,3-hexafluoropropane and 2-hydroxyethyl methacrylate or 2-aminoethyl methacrylate, or the corresponding acrylates, as illustrated in group D above, and (iv) other compounds containing at least one group or moiety capable of free radical polymerization and at least one aromatic ring or moiety containing electron-withdrawing substituents that do not interfere with free radical polymerization. The soft crosslinker is typically an diacrylic or dimethacrylic ester or ether of bisphenol A, but also include as soft crosslinkers are the other glycol dimethacrylates and diacrylates mentioned herein. Preferred soft crosslinkers are ethoxylated bisphenol A dimethacrylate and the adduct of glycidylmethacrylate and bisphenol A,

(e) The fluoride component is present in the coating as a component of a non-resinous component of the formulation. The fluoride component may be, but need not be soluble in the resin component of the coating. In the preferred practice of the invention, the fluoride component in the coating will dissolve in water and to the extent the water is removed from the fluoride source, fluoride is carried with it. The preferred form of the fluoride component, is an inorganic fluoride in which the fluoride is present, e.g., in the form of an fluorosilicate structure or an alumina fluoride structure. The fluoride source of the patent is a glass composition in which the fluoride content is derived from an alkaline earth metal fluoride such as calcium fluoride, barium fluoride and strontium fluoride. A most preferred fluoride source is described in U.S. Pat. No. 5,360,770, which is hereby incorporated by reference in its entirety. The coating is optionally provided with a leachable fluoride component. The fluoride is leachable from the coating over a three to four month period. This means that after many days and even months, the coating should be able to release small measured amounts of fluoride into the area surrounding the stent. The longevity of the fluoride in the coating and the ability to meter it from the coating are dependent on a number of factors, such as:

the concentration of fluoride in the coating;
the nature of the chemical bond of the fluoride within the coating composition;
the level of hygroscopicity of the coating;
if the fluoride is part of a solid, the degree of particulateness of the solid, coupled with the rate at which fluoride can be leached from the solid;
if the fluoride is part of a liquid molecule, the rate at which the fluoride is cleaved from the molecule to form a leachable fluoride; and
if the fluoride is part of a polymer, the rate at which fluoride in the polymer can be solubilized and leached from the polymer.

A particularly desirable form of the fluoride component, is an inorganic fluoride in which the fluoride is present, e.g., in the form of an fluorosilicate structure or an alumina fluoride structure. Illustrative of such fluoride structures are fluorite (or fluorspar), CaF², BaF₂, SrF₂, cryolite, Na3AlF₆, and fluorapatite, 3Ca₃(PO₄)₂ Ca(F,Cl)₂. A preferred fluoride source is described in U.S. Pat. No. 5,360,770. The fluoride source of the patent is a glass composition in which the fluoride content is derived from an alkaline earth metal fluoride such as calcium fluoride, barium fluoride and strontium fluoride. A particularly preferred glass composition that provides fluoride is the following:

TABLE 1

| Component | Mole % | Component | Mole % |
|---|---|---|---|
| SiO₂ | 17.6–21.6 | P₂O₅ | 0.8–3.5 |
| Al₂O₃ | 9.0–11.0 | Na₂O | 0.5–3.0 |
| MO | 7.9–19.7 | F | 42.2–56.1 |

in which M is an alkaline earth metal and MO is barium oxide and barium oxide binary and ternary mixtures with other alkaline earth metal oxides, such as BaO, BaO—CaO, BaO—SrO and CaO—BaO—SrO. Such preferred source of fluoride not only provides long term fluoride release from the coating but it also provides an essentially uniform release of fluoride over that period of time. FIGS. 1 and 2 illustrate the long term fluoride leachability of this fluoride source. FIG. 1 illustrates the release of fluoride by placing the aforementioned barium oxide based glass in water and determining the release of fluoride over an extended period of time. As can be seen, the fluoride release follows a straight line showing uniform release over 550 days, about 1½ years. FIG. 2 shows area plots of ingredients in order to optimize the glass formulation for maximizing the fluoride release over an extended period, e.g., 1½ years.

(f) Also included in the formulation, as an optional ingredient, is a photoinitiator. According to one aspect this invention, the light-initiated curing of a polymerizable matrix material involves photosensitization of light-sensitive compounds by ultraviolet or visible light, which, in turn, initiates polymerization of the matrix material. The photoinitiator to be used in this invention comprises a combination of a photosensitive ketone and a tertiary amine. Typical photosensitive ketones include benzophenone, acetophenone, thioxanthen-9-one, 9-fluorenone, anthraquinone, 4′-methoxyacetophenone, diethoxyacetophenone, biacetyl, 2,3-pentadione, benzyl, 4,4′-methoxybenzil, 4,4′-oxidibenzil, and 2,3-bornadione (dl camphroquinone). Typical tertiary amines include ethyl-4-dimethyl amino benzoate, ethyl-2-dimethyl amino benzoate, 4,4′-bis(dimethylamino) benzophenone, N-methyldiethanolamine, and dimethylaminobenzaldehyde. A preferred combination of the photoinitiators is 2,3-bornanedione with ethyl-4-dimethyl amino benzoate. Other suitable initiator are illustrated in U.S. Pat. No. 4,674,980 to Ibsen, et al., the disclosure of which is hereby incorporated by reference in its entirety. Alternatively, any known photosensitizing system which can function effectively in a paste/paste composition when exposed to light may substitute for the above-named compounds or combinations. The amount of the photoinitiator should be sufficient to initiate polymerization in a selected resin and complete it in depth within about half a minute when the filler-resin composition is exposed to a visible-light output of at least 5,000 foot candles. In addition, any known free-radical scavenger (anti-oxidants) such as butylated hydroxytoluene can be used to scavenge small amounts of free radicals generated during extended shelf storage.

(g) The polymerization system of the coating composition may depend on effecting cure with either the photoinitiator or by use of a thermal initiator, which is a typical thermal curing agent known in the art. Illustrative of these are benzoyl peroxide, dicumyl peroxide, ditertiary butyl peroxide, tertiary butyl hydroperoxide, cumyl hydroperoxide, or other suitable peroxides may initiate polymerization of the polymerizable ethylenically unsaturated components of the primary coating. Addition of such thermal initiators is desirable to insure complete polymerization. Even when light alone does not cure the matrix material, the peroxide initiates curing of the uncured material thermally upon standing. Benzoyl peroxide may be used together with 2-hydroxyethyl-p-toluidine.

The coating may contain pigments such as iron oxide or titanium oxide and a color stabilizing agent such as 2,2-hydroxy-5-tert. octyl phenylbenzotriazole.

In formulating the coating, the selection of the ingredients in formulating the coating is narrowly critical. Illustrative of such a formulation is the paste/paste coating composition as set forth in Table 2.

TABLE 2

| | |
|---|---|
| Ingredients | Percentage by Weight |

Paste A

| | |
|---|---|
| Glass, fluoride source | 0–85 |
| Ethylenically unsaturated monomer, e.g., 2- | 3–40 |
| hydroxyethyl methacrylate | |
| Soft Crosslinker, e.g., Ethoxylated bisphenol A | 10–60 |
| dimethacrylate | |
| 2,3-bornanedione | 0.03–0.30 |
| Butylated hydroxytoluene | 0.001–1.0 |
| Benzoyl peroxide | 0.005–0.10 |
| Polycarboxylic acid, e.g., polyacrylic acid | 0–8 |
| Hard Crosslinker, e.g., PMDM | 2–20 |
| d-Tartaric acid | 0–1 |
| 2,2-Hydroxy-5-tert-octyl phenylbenzotriazole | 0.00–2 |
| Ethyl 4-dimethylaminobenzoate | 0.00–2 |

Paste B

| | |
|---|---|
| Glass, fluoride source | 0–70 |
| Ethylenically unsaturated monomer, e.g., 2- | 0–45 |
| hydroxyethyl methacrylate | |
| Soft Crosslinker, e.g., ethoxylated bisphenol A | 10–90 |
| dimethacrylate | |
| Coupling agent, e.g., Na NTG-GMA, NGT-GMA | 1–20 |
| Zinc oxide | 0–15 |
| Barium tungstate | 0–15 |
| Ethyl 4-dimethylamino benzoate | 0–2.0 |
| 2,3-bornanedione | 0.05–0.30 |
| Butylated hydroxytoluene | 0.005–0.10 |
| Titanium dioxide | 0.0–3.0 |
| 2,2-Hydroxy-5-tert-octyl phenylbenzotriazole | 0.00–2 |

The two pastes, Paste A and Paste B, are preferably mixed well in equal amounts. The pastes may be mixed with a spatula or put onto a blade mixer prior to application to a surface. For example, the physician or technician may use the system by combining the pastes in the ratios desired, and then mixing them. The resulting paste is then applied to the surface as needed. The coating will self-cure in about 20-30 minutes, but cures instantly on exposure to light. Light having a wave length of about 480 nm at an intensity of about 5000 foot-candles is preferred. An exposure of about 30 second is sufficient to cure the cement in most applications.

A primer coating may be applied to the surface of the stent or the underlying drug-containing layer before coating on the primary coating. This may be effected by the following procedure:

(1) First contacting the surface with an aqueous solution comprising at least one strong acid or acidic salt with a dispensable brush or a skube (a preformed Styrofoam™ sponge) in order to condition the surface, allow to absorb for 15 seconds and blot dry with a skube. Note: if hemorrhage is in the area, use a hemostatic solution or the aqueous solution with a hemostatic solution to control seepage and keep the bonding surface dry.

(2) Immediately mix with stirring with a dispensable brush a solution comprising a solvent and at least one compound selected from the group consisting of (1) N-phenylglycine, (2) the adduct of N-(p-tolyl)glycine and glycidyl methacrylate, (3) the addition reaction product of N-phenylglycine and glycidyl methacrylate, and (4) other amino acids, in which each member of the group of (1), (2), (3) and (4) that is present in the solution is an alkali metal salt form of that member, and a solution comprising at least one monomer selected from the group consisting of (1) the addition reaction product of pyromellitic acid dianhydride and 2-hydroxyethyl methacrylate, (2) the addition reaction product of 3,3′, 4,4′-benzophenone tetracarboxylic dianhydride and 2-hydroxyethyl methacrylate, (3) 4-methacryloxyethyltrimellitic-anhydride, and (4) other compounds containing at least one group or moiety capable of free radical polymerization and at least one aromatic ring or moiety containing electron-withdrawing substituents that do not interfere with free radical polymerization. Apply 3-5 coats of the mixture onto the prepared bonding surface with the dispensable brush used for mixing. Allow to dry for 15 seconds.

(3) Mix Paste A and B together and load into a syringe. Immediately inject the paste mixture onto the prepared bonding surface and light-activate for 30 seconds. This will effect cure.

The above procedure can be effected without using the primer coating. In such an embodiment, it is important to clean the surface to which the primary coating is being applied. Water washing the surface if an acid wash is not recommended or needed will prepare the surface provided the surface is thoroughly dry before applying the primary coating.

The primer coating may contain solvent solutions of the free acid or alkali metal salt of (i) N-phenylglycine, (ii) the adduct of N—(P-tolyl)glycine and glycidyl methacrylate, which are illustrated by the structures:

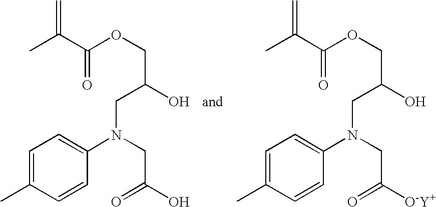

where Y is one of the alkali metals, i.e., lithium, sodium, potassium, rubidium and cesium, preferably sodium or potassium, and (iii) the adduct of N-phenylglycine and glycidyl methacrylate, the alkali metal salt thereof, or the mixture of the foregoing two compounds, which compounds are illustrated by the structures, and (iii) the adduct of N-phenylglycine and glycidyl methacrylate, which are illustrated by the structures:

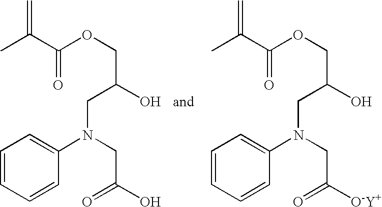

where Y is described above; and the solvent solution of PMDM (see the isomeric mixture of “B” above that describes the adduct of pyromellitic acid dianhydride and 2-hydroxyethyl methacrylate). The preferred solvent is a mixture of water and a polar solvent such as acetone.

When applying the primer coating, the surface may be prepared with an acid wash. The first stage of the primer coating may be a solvent solution of the NTG-GMA adduct, typically dried before the second solution is applied to it. The second stage is a solution of, e.g., PMDM that is coated over the first stage. That coating is also dried before applying the primary coating. On drying, the primer coating is cured. Drying may be effected at ambient conditions, or accelerated by the addition of heat to the undried coating.

The different coatings can be applied to the stent by any common coating methods. The therapeutic substance containing layer is commonly applied by preparing a solution comprising a solvent, a polymer dissolved in the solvent, and a therapeutic drug dispersed in the solvent; applying the solution to the stent body by any method; and evaporating off the solvent to leave the coating. The application to the stent body may be accomplished by dipping the stent in the solution, spraying the solution on the stent, or any other coating method such as chemical vapor deposition or plasma deposition. The amount of therapeutic substance incorporated on the stent body can be controlled by making several applications of the solution allowing the coating to dry to a layer after each application. The polymer of the solution may be any polymer that allows the drug to elute over time. The polymer may also be the cross-linked resin that makes up the primary coating of the biocompatible layer. One method of forming a therapeutic substance layer on a stent and the layer itself is described in U.S. Pat. No. 5,599,352, issued to Dinh et. al, which is hereby incorporated by reference in its entirety.

The cross-linked resin layer may be applied by any known method and as described above a primer layer may be used to adhere the cross-linked resin layer to the stent. Exemplary methods of application include brushing, using a syringe, spraying, dip-coating, chemical vapor deposition, and plasma deposition. These methods may also be used to apply the optional primer layer. The cross-linked resin layer may be applied by the physician at the time of surgery as described in U.S. Pat. No. 5,876,743 or at any time before surgery in a batch or continuous process. The drug containing layer will normally be applied before the stent reaches the physician but could also be applied by the physician at the time of surgery.

Although the above description includes details about specific embodiments, these details are not meant to limit the scope or breadth of the invention in any way. Other embodiments and methods that are obvious variations of the embodiments and methods disclosed herein are intended to be encompassed by the invention. The invention is meant only to be limited by the appended claims.

What is claimed is:

1. A biocompatible drug eluting stent comprising:
a stent body;
a first layer applied over the stent body comprising a therapeutic substance; and
a second layer applied over the first layer comprising a fluoride-containing hydrophilic water insoluble crosslinked resin.

2. The stent of claim 1 wherein the therapeutic substance is selected from the group consisting of anticoagulant drugs, antiplatelet drugs, antimetabolite drugs, anti-inflammatory drugs and antimitotic drugs.

3. The stent of claim 2 wherein the therapeutic substance is selected from the group consisting of glucocorticoids, dexamethasone, betamethasone, heparin, hirudin, tocopherol, angiopeptin, aspirin, ACE inhibitors, growth factors, and oligonucleotides.

4. The stent of claim 1 wherein the second layer leaches fluoride over a period of time.

5. The stent of claim 1 wherein the second layer comprises Geristore™.

6. The stent of claim 1 wherein the second layer comprises a two component blend in which the
a) first component comprises:
i) a fluoride source that includes a particulate siliceous fluoride containing filler in which the fluoride is water leachable;
ii) a coupling agent that includes one or more of (i) N-phenylglycine, the alkali metal salt thereof, or the mixture of the foregoing two compounds, (ii) the adduct of N-(p-tolyl)glycine and glycidyl methacrylate, the alkali metal salt thereof, or the mixture of the foregoing two compounds, and (iii) the adduct of N-phenylglycine and glycidyl methacrylate, the alkali metal salt thereof, or the mixture of the foregoing two compounds;
iii) a photoinitiator; optionally, a radiopaquing agent; and, optionally, a buffering agent; and
b) second component comprises:
i) an ethylenically-unsaturated-functional monomer;
ii) a soft crosslinker that includes one or more of 2,2-bis (4-methacryloxy 2-ethoxyphenyl) propane and diethyleneglycol bismethacrylate;
iii) a hard crosslinker that includes one or more of (i) the adduct of pyromellitic acid dianhydride and 2-hydroxyethylmethacrylate, (ii) the adduct of 3,3′,4,4′-benzophenonetetracarboxylic dianhydride and 2-hydroxyethylmethacrylate, (iii) 4-methacryloxyethyltrimellitic anhydride, and (iv) other compounds containing at least one group or moiety capable of free radical polymerization and at least one aromatic ring or moiety containing electron withdrawing substituents that do not interfere with free radical polymerization;
iv) a photoinitiator;
v) a polymerized carboxylic acid;
vi) a free-radical scavenger; and
vii) a curing catalyst.

7. The stent of claim 1 wherein the second layer comprises a light-curable adhesive composition containing:
a) a first component comprising:
i) a fluoride source including a particulate siliceous fluoride containing filler in which the fluoride is water leachable;
ii) a soft crosslinker;
iii) an ethylenically-unsaturated-functional monomer;
iv) a photoinitiator;
v) a free-radical scavenger;
vi) a thermal initiator;
vii) a polymerized carboxylic acid;
viii) a hard crosslinker including one or more of (i) the adduct of pyromellitic acid dianhydride and 2-hydroxyethylmethacrylate; (ii) the adduct of 3,3′,4,4′-benzophenonetetracarboxylic dianhydride and 2-hydroxyethylmethacrylate, (iii) 4-methacryloxyethyltrimellitic anhydride, and (iv) other compounds containing at least one group or moiety capable of free radical polymerization and at least one aromatic ring or moiety containing electron withdrawing substituents that do not interfere with free radical polymerization, and
b) a second component comprising:
i) a fluoride source including a particulate siliceous fluoride containing filler in which the fluoride is water leachable;
ii) a soft crosslinker;
iii) an ethylenically-unsaturated-functional monomer;
iv) a coupling agent including one or more of (i) N-phenylglycine, the alkali metal salt thereof, or the mixture of the foregoing two compounds, (ii) the adduct of N-(p-tolyl)glycine and glycidyl methacrylate, the alkali metal salt thereof, or the mixture of the foregoing two compounds, and (iii) the adduct of N-phenylglycine and glycidyl methacrylate, the alkali metal salt thereof, or the mixture of the foregoing two compounds;
v) a photoinitiator;
vi) a radiopaquing agent; and
vii) a buffering agent.

8. The stent of claim 1 wherein the second layer comprises contains:
a) a particulate glass having the composition of

| Component | Mole % | Component | Mole % |
|---|---|---|---|
| SiO₂ | 17.6–21.6 | P₂O₅ | 0.8–3.5 |
| Al₂O₃ | 9.0–11.0 | Na₂O | 0.5–3.0 |
| MO | 7.9–19.7 | F | 42.2–56.1 |

in which M is an alkaline earth metal and MO is barium oxide and barium oxide binary and ternary mixtures with other alkaline earth metal oxides;
b) the alkali metal salt of the adduct of N-(p-tolyl)glycine and glycidyl methacrylate;
c) the adduct of pyromellitic acid dianhydride and 2-hydroxyethyl methacrylate;
d) ethyl 4-dimethylamino benzoate and camphoquinone (i.e., 2, 3-bornanedione);
e) ethoxylated bisphenol A dimethacrylate and the adduct of glycidylmethacrylate and bisphenol A,
f) 2-hydroxyethyl methacrylate;
g) butylated hydroxytoluene free radical scavenger
h) polyacrylic acid; and
i) benzoyl peroxide or other peroxide that cause free radical addition at about 55° C. or at a lower temperature.

9. A method for forming a biocompatible drug eluting stent comprising:
providing a stent body;
applying a first layer to the stent body comprising a therapeutic substance; and
applying over the first layer a second layer comprising a fluoride-containing hydrophilic water insoluble crosslinked resin.

10. The method of claim 9 wherein the step of applying the first layer comprises applying a layer comprising at least one therapeutic substance selected from the group consisting of anticoagulant drugs, antiplatelet drugs, antimetabolite drugs, anti-inflammatory drugs and antimitotic drugs.

11. The method of claim 9 wherein the step of applying the first layer comprises applying a layer comprising at least one therapeutic substance selected from the group consisting of glucocorticoids, dexamethasone, betamethasone, heparin, hirudin, tocopherol, angiopeptin, aspirin, ACE inhibitors, growth factors, and oligonucleotides.

12. The method of claim 9 wherein the step of applying the second layer comprises applying a layer that leaches fluoride over a period of time.

13. The method of claim 9 wherein the step of applying the second layer comprises applying a layer comprising Geristore™.

14. The method of claim 9 wherein step applying the second layer comprises at least one process selected from the group consisting of brush application, syringe application, spraying, dip-coating, chemical vapor deposition and plasma deposition.

15. A method for forming a biocompatible stent comprising:
providing a stent body;
applying a fluoride-containing hydrophilic water insoluble crosslinked resin layer over the stent body.

16. The method of claim 15 wherein the step of applying the fluoride-containing hydrophilic water insoluble crosslinked resin layer comprises applying a layer that leaches fluoride over a period of time.

17. The method of claim 15 wherein the step of applying the fluoride-containing hydrophilic water insoluble crosslinked resin layer comprises applying a layer comprising Geristore™.

18. The method of claim 15 further comprising applying a primer layer to the stent body before applying the fluoride-containing hydrophilic water insoluble crosslinked resin layer.

19. The method of claim 15 wherein the step of applying the fluoride-containing hydrophilic water insoluble crosslinked resin layer comprises a process selected from the group consisting of brush application, syringe application, spraying, dip-coating, chemical vapor deposition and plasma deposition.

20. A biocompatible drug eluting stent comprising:
a stent body;
an inner layer applied over the stent body comprising a fluoride-containing hydrophilic water insoluble crosslinked resin;
an intermediate layer over the inner layer comprising a therapeutic substance; and
an outer layer over the intermediate layer comprising a fluoride-containing hydrophilic water insoluble crosslinked resin.

21. The stent of claim 20 wherein the therapeutic substance is selected from the group consisting of anticoagulant drugs, antiplatelet drugs, antimetabolite drugs, anti-inflammatory drugs and antimitotic drugs.

22. The stent of claim 20 wherein the therapeutic substance is selected from the group consisting of glucocorticoids, dexamethasone, betamethasone, heparin, hirudin, tocopherol, angiopeptin, aspirin, ACE inhibitors, growth factors, and oligonucleotides.

23. The stent of claim 20 wherein at least the outer layer leaches fluoride over a period of time.

24. The stent of claim 20 wherein at least one of the inner layer and outer layer comprises Geristore™.

25. A biocompatible drug eluting stent comprising:
a stent body;
a covering on the stent body comprising a therapeutic substance incorporated into a fluoride-containing hydrophilic water insoluble crosslinked resin.

26. The stent of claim 25 wherein the therapeutic substance is selected from the group consisting of anticoagulant drugs, antiplatelet drugs, antimetabolite drugs, anti-inflammatory drugs and antimitotic drugs.

27. The stent of claim 25 wherein the therapeutic substance is selected from the group consisting of glucocorticoids, dexamethasone, betamethasone, heparin, hirudin, tocopherol, angiopeptin, aspirin, ACE inhibitors, growth factors, and oligonucleotides.

28. The stent of claim 25 wherein the fluoride-containing hydrophilic water insoluble crosslinked resin leaches fluoride over a period of time.

29. The stent of claim 25 wherein the fluoride-containing hydrophilic water insoluble crosslinked resin comprises Geristore™.
